SEQUENCE LISTING (16 sequences)

SEQ ID NO: 1 - DNA, 27 bp, synthetic construct
  source 1..27: mol_type = other DNA
agcttcatagtttgaaaaacgtgcacc

SEQ ID NO: 2 - DNA, 27 bp, synthetic construct
  source 1..27: mol_type = other DNA
gctttgcaaaaggcagacaaatttacc

SEQ ID NO: 3 - DNA, 21 bp, synthetic construct
  source 1..21: mol_type = other DNA
aagaagctgtaaccgtacccc

SEQ ID NO: 4 - DNA, 21 bp, synthetic construct
  source 1..21: mol_type = other DNA
agaccgtgtaattcgtcaccg

SEQ ID NO: 5 - DNA, 11060 bp, synthetic construct
  source 1..11060: mol_type = other DNA
gctcacgtgacgcgtctcgagaagctttcctgtgtgaaattgttatccgctcgccggcggctgctaacaaagcccgaaaggaagctgagttggctgctgccaccgctgagcaataactagcataaccccttggggcctctaaacgggtcttgaggggttttttgctgaaaggaggaactatatccggatgaccgacctgtccggtgccctgaatgaactgcaggacgagtaatcacactggctcaccttcgggtgggcctttctgcgtttatgctagtgataataagtgactgaggtatgtgctcttcttatctccttttgtagtgttgctcttattttaaacaactttgcggttttttgatgactttgcgattttgttgttgctttgcagtaaattgcaagatttaataaaaaaacgcaaagcaatgattaaaggatgttcagaatgaaactcatggaaacacttaaccagtgcataaacgctggtcatgaaatgacgaaggctatcgccattgcacagtttaatgatgacagcccggaagcgaggaaaataacccggcgctggagaataggtgaagcagcggatttagttggggtttcttctcaggctatcagagatgccgagaaagcagggcgactaccgcacccggatatggaaattcgaggacgggttgagcaacgtgttggttatacaattgaacaaattaatcatatgcgtgatgtgtttggtacgcgattgcgacgtgctgaagacgtatttccaccggtgatcggggttgctgcccataaaggtggcgtttacaaaacctcagtttctgttcatcttgctcaggatctggctctgaaggggctacgtgttttgctcgtggaaggtaacgacccccagggaacagcctcaatgtatcacggatgggtaccagatctcatgattcatgcagaagacactctcctgcctttctatcttggggaaaaggacgatgtcacttatgcaataaagcccacttgctggccggggcttgacattattccttcctgtctggctctgcaccgtattgaaactgagttaatgggcaaatttgatgaaggtaaactgcccaccgatccacacctgatgctccgactggccattgaaactgttgctcatgactatgatgtcatagttattgacagcgcgcctaacctgggtatcggcacgattaatgtcgtatgtgctgctgatgtgctgattgttcccacgcctgctgagttgtttgactacacctccgcactgcagtttttcgatatgcttcgtgatctgctcaagaacgttgatcttaaagggttcgagcctgatgtacgtattttgcttaccaaatacagcaatagtaatggctctcagtccccgtggatggaggagcaaattcgggatgcctggggaagcatggttctaaaaaatgttgtacgtgaaacggatgaagttggtaaaggtcagatccggatgagaactgtttttgaacaggccattgatcaacgctcttcaactggtgcctggagaaatgctctttctatttgggaacctgtctgcaatgaaattttcgatcgtctgattaaaccacgctgggagattagataatgaagcgtgcgcctgttattccaaaacatacgctcaatactcaaccggttgaagatacttcgttatcgacaccagctgccccgatggtggattcgttaattgcgcgcgtaggagtaatggctcgcggtaatgccattactttgcctgtatgtggtcgggatgtgaagtttactcttgaagtgctccggggtgatagtgttgagaagacctctcgggtatggtcaggtaatgaacgtgaccaggagctgcttactgaggacgcactggatgatctcatcccttcttttctactgactggtcaacagacaccggcgttcggtcgaagagtatctggtgtcatagaaattgccgatgggagtcgccgtcgtaaagctgctgcacttaccgaaagtgattatcgtgttctggttggcgagctggatgatgagcagatggctgcattatccagattgggtaacgattatcgcccaacaagtgcttatgaacgtggtcagcgttatgcaagccgattgcagaatgaatttgctggaaatatttctgcgctggctgatgcggaaaatatttcacgtaagattattacccgctgtatcaacaccgccaaattgcctaaatcagttgttgctcttttttctcaccccggtgaactatctgcccggtcaggtgatgcacttcaaaaagcctttacagataaagaggaattacttaagcagcaggcatctaaccttcatgagcagaaaaaagctggggtgatatttgaagctgaagaagttatcactcttttaacttctgtgcttaaaacgtcatctgcatcaagaactagtttaagctcacgacatcagtttgctcctggagcgacagtattgtataagggcgataaaatggtgcttaacctggacaggtctcgtgttccaactgagtgtatagagaaaattgaggccattcttaaggaacttgaaaagccagcaccctgatgcgaccacgttttagtctacgtttatctgtctttacttaatgtcctttgttacaggccagaaagcataactggcctgaatattctctctgggcccactgttccacttgtatcgtcggtctgataatcagactgggaccacggtcccactcgtatcgtcggtctgattattagtctgggaccacggtcccactcgtatcgtcggtctgattattagtctgggaccacggtcccactcgtatcgtcggtctgataatcagactgggaccacggtcccactcgtatcgtcggtctgattattagtctgggaccatggtcccactcgtatcgtcggtctgattattagtctgggaccacggtcccactcgtatcgtcggtctgattattagtctggaaccacggtcccactcgtatcgtcggtctgattattagtctgggaccacggtcccactcgtatcgtcggtctgattattagtctgggaccacgatcccactcgtgttgtcggtctgattatcggtctgggaccacggtcccacttgtattgtcgatcagactatcagcgtgagactacgattccatcaatgcctgtcaagggcaagtattgacatgtcgtcgtaacctgtagaacggagtaacctcggtgtgcggttgtatgcctgctgtggattgctgctgtgtcctgcttatccacaacattttgcgcacggttatgtggacaaaatacctggttacccaggccgtgccggcacgttaaccgggctgcatccgatgcaagtgtgtcgctgtcgacgagctcgcgagctcggacatgaggttgccccgtattcagtgtcgctgatttgtattgtctgaagttgtttttacgttaagttgatgcagatcaattaatacgatacctgcgtcataattgattatttgacgtggtttgatggcctccacgcacgttgtgatatgtagatgataatcattatcactttacgggtcctttccggtgatccgacaggttacggggcggcgacctcgcgggttttcgctatttatgaaaattttccggtttaaggcgtttccgttcttcttcgtcataacttaatgtttttatttaaaataccctctgaaaagaaaggaaacgacaggtgctgaaagcgagctttttggcctctgtcgtttcctttctctgtttttgtccgtggaatgaacaatggaagtccgagctcatcgctaataacttcgtatagcatacattatacgaagttatattcgatgctgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttcctgccactcatcgcagtactgttgtaattcattaagcatgcagaaagtcaaaagcctccgaccggaggcttttgactattactgccgacatggaagccatcacaaacggcatgatgaacctataaacgcagaaaggcccacccgaaggtgagccagtgtgattaccagcggcatcagcaccttgtcgccttgcgtataatatttgcaaaaaacccctcaagacccgtttagaggccccaaggggttatgctaggcccgggcgtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgactaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtctaagaaaccattattatcatgacattaacctataaaaataggcgtatcacgaggccctttaatctcgcgcgtttcggtgatgacggtgaaaacctctgacacatgcagctcccggagattgtcacagcttgtctgtaagcggatgccgggagcagacaagcccgtcagggcgcgtcagcgggtgttggcgggtgtcggggctggcttaactatgcggcatcagagcagattgtactgagagtgcaccatatcgactacgtcgttaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatataaattaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcattagttttgctggccgcatcttctcaaatatgcttcccagcctgcttttctgtaacgttcaccctctaccttagcatcccttccctttgcaaatagtcctcttccaacaataataatgtcagatcctgtagagaccacatcatccacggttctatactgttgacccaatgcgtctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagatagggagcccttgcatgacaattctgctaacatcaaaaggcctctaggttcctttgttacttcttctgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggaaaaatcagtcaagatatccacatgtgtttttagtaaacaaattttgggacctaatgcttcaactaactccagtaattccttggtggtacgaacatccaatgaagcacacaagtttgtttgcttttcgtgcatgatattaaatagcttggcagcaacaggactaggatgagtagcagcacgttccttatatgtagctttcgacatgatttatcttcgtttcctgcaggtttttgttctgtgcagttgggttaagaatactgggcaatttcatgtttcttcaacactacatatgcgtatatataccaatctaagtctgtgctccttccttcgttcttccttctgttcggagattaccgaatcaaaaaaatttcaaagaaaccgaaatcaaaaaaaagaataaaaaaaaaatgatgaattgaattgaaaagctgtggtatggtgcactctcagtacaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtggatatactagaagttctcctcgaccgtcgatatgcggtgtgaaataccgcacagatgctaccgctgaaagttctgcaaagcctgatgggacataagtccatcagttcaacggaagtctacacgaaggtttttgcgctggatgtggctgcccggcaccgggtgcagtttgcgatgccggagtctgatgcggttgcgatgctgaaacaattatcctgagaataaatgccttggcctttatatggaaatgtggaactgagtggatatgctgtttttgtctgttaaacagagaagctggctgttatccactgagaagcgaacgaaacagtcgggaaaatctcccattatcgtagagatccgcattattaatctcaggagcctgtgtagcgtttataggaagtagtgttctgtcatgatgcctgcaagcggtaacgaaaacgatttgaatatgccttcaggaacaatagaaatcttcgtgcggtgttacgttgaagtggagcggattatgtcagcaatggacagaacaacctaatgaacacagaaccatgatgtggtctgtccttttacagccagtagtgctcgccgcagtcgagcgacagggcgaagccctcggctggttgccctcgccgctgggctggcggccgtctatggccctgcaaacgcgccagaaacgccgtcgaagccgtgtgcgagacaccgcggccggccgccggctttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggtcgagtgagcgaggaagcaccagggaacagcacttatatattctgcttacacacgatgcctgaaaaaacttcccttggggttatccacttatccacggggatatttttataattattttttttatagtttttagatcttcttttttagagcgccttgtaggcctttatccatgctggttctagagaaggtgttgtgacaaattgccctttcagtgtgacaaatcaccctcaaatgacagtcctgtctgtgacaaattgcccttaaccctgtgacaaattgccctcagaagaagctgttttttcacaaagttatccctgcttattgactcttttttatttagtgtgacaatctaaaaacttgtcacacttcacatggatctgtcatggcggaaacagcggttatcaatcacaagaaacgtaaaaatagcccgcgaatcgtccagtcaaacgacctcactgaggcggcatatagtctctcccgggatcaaaaacgtatgctgtatctgttcgttgaccagatcagaaaatctgatggcaccctacaggaacatgacggtatctgcgagatccatgttgctaaatatgctgaaatattcggattgacctctgcggaagccagtaaggatatacggcaggcattgaagagtttcgcggggaaggaagtggttttttatcgccctgaagaggatgccggcgatgaaaaaggctatgaatcttttccttggtttatcaaacgtgcgcacagtccatccagagggctttacagtgtacatatcaacccatatctcattcccttctttatcgggttacagaaccggtttacgcagtttcggcttagtgaaacaaaagaaatcaccaatccgtatgccatgcgtttatacgaatccctgtgtcagtatcgtaagccggatggctcaggcatcgtctctctgaaaatcgactggatcatagagcgttaccagctgcctcaaagttaccagcgtatgcctgacttccgccgccgcttcctgcaggtctgtgttaatgagatcaacagcagaactccaatgcgcctctcatacattgagaaaaagaaaggccgccagacgactcatatcgtattttccttccgcgatatcacttccatgacgacaggatagtctgagggttatctgtcacagatttgagggtggttcgtcacatttgttctgacctactgagggtaatttgtcacagttttgctgtttccttcagcctgcatggattttctcatactttttgaactgtaatttttaaggaagccaaatttgagggcagtttgtcacagttgatttccttctctttcccttcgtcatgtgacctgatatcgggggttagttcgtcatcattgatgagggttgattatcacagtttattactctgaattggctatccgcgtgtgtacctctacctggagtttttcccacggtggatatttcttcttgcgctgagcgtaagagctatctgacagaacagttcttctttgcttcctcgccagttcgctcgctatgctcggttacacggctgcggcgagctgcctggcggcagtagcgcggtggtcccacctgaccccatgccgaactcagaagtgaaacgccgtagcgccgatggtagtgtggggtctccccatgcgagagtagggaactgccaggcatcaaataaaacgaaaggctcagtcgaaagactgggccttggattgcacgcaggttctccggccgcttgggtggagaggcgcagaaagtcaaaagcctccgaccggaggcttttgactattaggctatgactgggcacaacagacaatcggctgctctgatgagccgccagggttttcccagtcacgacggatccgaattcagc

SEQ ID NO: 6 - DNA, 4384 bp, synthetic construct
  source 1..4384: mol_type = other DNA
aggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataccacagcttttcaattcaattcatcattttttttttattcttttttttgatttcggtttctttgaaatttttttgattcggtaatctccgaacagaaggaagaacgaaggaaggagcacagacttagattggtatatatacgcatatgtagtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcagaagaagtaacaaaggaacctagaggccttttgatgttagcagaattgtcatgcaagggctccctatctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattaccctatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacataggagccggaagcataaagtgtaaagcctggggtgcctaatgagtgaggtaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacg

SEQ ID NO: 7 - DNA, 5407 bp, synthetic construct
  source 1..5407: mol_type = other DNA
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatagccatcctcatgaaaactgtgtaacataataaccgaagtgtcgaaaaggtggcaccttgtccaattgaacacgctcgatgaaaaaaataagatatatataaggttaagtaaagcgtctgttagaaaggaagtttttcctttttcttgctctcttgtcttttcatctactatttccttcgtgtaatacagggtcgtcagatacatagatacaattctattacccccatccatacaatgccatctcatttcgatactgttcaactacacgccggccaagagaaccctggtgacaatgctcacagatccagagctgtaccaatttacgccaccacttcttatgttttcgaaaactctaagcatggttcgcaattgtttggtctagaagttccaggttacgtctattcccgtttccaaaacccaaccagtaatgttttggaagaaagaattgctgctttagaaggtggtgctgctgctttggctgtttcctccggtcaagccgctcaaacccttgccatccaaggtttggcacacactggtgacaacatcgtttccacttcttacttatacggtggtacttataaccagttcaaaatctcgttcaaaagatttggtatcgaggctagatttgttgaaggtgacaatccagaagaattcgaaaaggtctttgatgaaagaaccaaggctgtttatttggaaaccattggtaatccaaagtacaatgttccggattttgaaaaaattgttgcaattgctcacaaacacggtattccagttgtcgttgacaacacatttggtgccggtggttacttctgtcagccaattaaatacggtgctgatattgtaacacattctgctaccaaatggattggtggtcatggtactactatcggtggtattattgttgactctggtaagttcccatggaaggactacccagaaaagttccctcaattctctcaacctgccgaaggatatcacggtactatctacaatgaagcctacggtaacttggcatacatcgttcatgttagaactgaactattaagagatttgggtccattgatgaacccatttgcctctttcttgctactacaaggtgttgaaacattatctttgagagctgaaagacacggtgaaaatgcattgaagttagccaaatggttagaacaatccccatacgtatcttgggtttcataccctggtttagcatctcattctcatcatgaaaatgctaagaagtatctatctaacggtttcggtggtgtcttatctttcggtgtaaaagacttaccaaatgccgacaaggaaactgacccattcaaactttctggtgctcaagttgttgacaatttaaagcttgcctctaacttggccaatgttggtgatgccaagaccttagtcattgctccatacttcactacccacaaacaattaaatgacaaagaaaagttggcatctggtgttaccaaggacttaattcgtgtctctgttggtatcgaatttattgatgacattattgcagacttccagcaatcttttgaaactgttttcgctggccaaaaaccatgagtgtgcgtaatgagttgtaaaattatgtataaacctactttctctcacaagttatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtaccgggccccccctcgaggtcgacggtatcgataagcttgatatcgaattcctgcagcccgggggatccactagttctagagcggccgccaccgcggtggagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtc

SEQ ID NO: 8 - DNA, 3006 bp, synthetic construct
  source 1..3006: mol_type = other DNA
attgaatatcacgcttaataaaggagaataaatcgttttctactttcttctgctgctataataagcacctatgggatctatatagtatttttataacgatagactttataaaagaaaatacctaagtgaaaatttggtgaattttgagataattgttgggattccatttttaataaggcaataatattaggtatgtagaatatactagaagttctcctcgaggatttaggaatccataaaagggaatctgcaattctacacaattctataaatattattatcatcgttttatatgttaatattcattgatcctattacattatcaatccttgcgtttcagcttccactaatttagatgactatttctcatcatttgcgtcatcttctaacaccgtatatgataatatactagtaacgtaaatactagttagtagatgatagttgatttttattccaacactaagaaataatttcgccatttcttgaatgtatttaaagatatttaatgctataatagacatttaaatccaattcttccaacatacaatgggagtttggccgagtggtttaaggcgtcagatttaggtggatttaacctctaaaatctctgatatcttcggatgcaagggttcgaatcccttagctctcagacatggaggcccagaataccctccttgacagtcttgacgtgcgcagctcaggggcatgatgtgactgtcgcccgtacatttagcccatacatccccatgtataatcatttgcatccatacattttgatggccgcacggcgcgaagcaaaaattacggctcctcgctgcagacctgcgagcagggaaacgctcccctcacagacgcgttgaattgtccccacgccgcgcccctgtagagaaatataaaaggttaggatttgccactgaggttcttctttcatatacttccttttaaaatcttgctaggatacagttctcacatcacatccgaacataaacaaccatgggtaaggaaaagactcacgtttcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggcaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagtactgacaataaaaagattcttgttttcaagaacttgtcatttgtatagtttttttatattgtagttgttctattttaatcaaatgttagcgtgatttatattttttttcgcctcgacatcatctgcccagatgcgaagttaagtgcgcagaaagtaatatcatgcgtcaatcgtatgtgaatgctggtcgctatactggtgtgcgtaatgagttgtaaaattatgtataaacctactttctctcacaagtactatacttttataaaacgaactttattgaaatgaatatcctttttttcccttgttacatgtcgtgactcgtactttgaacctaaattgttctaacatcaaagaacagtgttaattcgcagtcgagaagaaaaatatggtgaacaagactcatctacttcatgagactactttacgcctcctataaagctgtcacactggataaatttattgtaggaccaagttacaaaagaggatgatggaggtttctttacaataaagaagcacatgtgtgttaacgtttttagtatttgcttgttatgtaaatcaggaaaacttcgcgggatttggttggatgctactttccatacaataaatattatagatctaaaaagccaaattacaagtaaagattagtaaagctgttggaattccatcgttgataaaaatgttagttattaaatataaaagtcagaataggtgaacttggatttaattgttggcatttcgttgctgctagaggccataatattagatagccaggacatactagttctcctcgtggtataggaatccataaaatggaattggtgattctatgtgatatattcacattcttactacattatcaatccttgcacttcagcttcctctaacctcgatgacatcttctcataacttatgtcatcatctaacgccgtctattataatatattgatagtataagtattagttgatagacaatagtggatttttattccaacagtgtctttgttcgtctcagatatagtcggattgcccttttaagcaatcaatagtgttttatttgcaacaatgtcgtcatagtttaatatgtcctataagatgttaacttgctcaacattcaacaaagtttggtctcttggcccagttggttaaggcaccgtgctaataacgcggggatcagcggttcgatcccgctagagaccat

SEQ ID NO: 9 - DNA, 60 bp, synthetic construct
  source 1..60: mol_type = other DNA
gatatcttcggatgcaagggttcgaatcccttagctctcagacatggaggcccagaatac

SEQ ID NO: 10 - DNA, 60 bp, synthetic construct
  source 1..60: mol_type = other DNA
aagtaggtttatacataattttacaactcattacgcacaccagtatagcgaccagcattc

SEQ ID NO: 11 - DNA, 20 bp, synthetic construct
  source 1..20: mol_type = other DNA
actgccgagcagcttgttat

SEQ ID NO: 12 - DNA, 20 bp, synthetic construct
  source 1..20: mol_type = other DNA
cacaacacgaggtacgggaa

SEQ ID NO: 13 - DNA, 21 bp, synthetic construct
  source 1..21: mol_type = other DNA
gccatccgccccatctaataa

SEQ ID NO: 14 - DNA, 20 bp, synthetic construct
  source 1..20: mol_type = other DNA
ccttccacgagcaaaacacg

SEQ ID NO: 15 - DNA, 20 bp, synthetic construct
  source 1..20: mol_type = other DNA
gagggtggttcgtcacattt

SEQ ID NO: 16 - DNA, 19 bp, synthetic construct
  source 1..19: mol_type = other DNA
taccgccactgcctatgtt

(19) United States
(12) Patent Application Publication
(10) Pub. No.: US 2025/0092407 A1
(43) Pub. Date: Mar. 20, 2025

(54) METHOD FOR SYNTHESIS, ASSEMBLY AND FUNCTION TEST OF ARTIFICIAL CHLOROPLAST GENOME OF CHLAMYDOMONAS REINHARDTII

(71) Applicant: SHENZHEN UNIVERSITY, Shenzhen, Guangdong (CN)

(72) Inventors: Zhangli HU, Shenzhen (CN); Chaogang WANG, Shenzhen (CN); Chunli GUO, Shenzhen (CN); Guiying ZHANG, Shenzhen (CN); Bin JIA, Shenzhen (CN)

(73) Assignee: SHENZHEN UNIVERSITY, Shenzhen, Guangdong (CN)

(21) Appl. No.: 18/559,847

(22) PCT Filed: Aug. 12, 2022
(86) PCT No.: PCT/CN2022/112271

§ 371 Date: Nov. 9, 2023

(30) Foreign Application Priority Data
Aug. 1, 2022 (CN) 202210917993.3

(51) Int. Cl. C12N 15/81; C12N 15/82; C12N 15/90; C12Q 1/6895; G01N 33/569

(52) U.S. Cl. CPC C12N 15/81; C12N 15/8207; C12N 15/905; C12Q 1/6895; G01N 33/56961; C12Q 2600/156; G01N 2333/405; G01N 2474/10

(57) ABSTRACT

A method for synthesis, assembly and function test of an artificial chloroplast genome of Chlamydomonas reinhardtii. Rational design has been carried out on the Chlamydomonas reinhardtii chloroplast genome for the first time, and total artificial synthesis of the Chlamydomonas reinhardtii chloroplast genome is proposed. By using totally chemically synthesized chloroplast genome segments, total chemical de novo synthesis and assembly of a chloroplast genome are achieved in a yeast-bacterium system. Then, a totally chemically synthesized chloroplast genome is transformed into Chlamydomonas cells to replace the original chloroplast genome, which works normally, and has been verified, fulfilling biological functions of the totally chemically synthesized chloroplast genome. According to the embodiments, the Chlamydomonas reinhardtii chloroplast genome is an efficient platform for carrying out synthetic biology operation.

### FIELD

The present invention relates to the field of synthetic biology, in particular, to a method for synthesis, assembly and function test of an artificial chloroplast genome of Chlamydomonas reinhardtii.

### BACKGROUND

Chlamydomonas reinhardtii is a single-cell eukaryotic algal model organism, which has three sets of genetic systems including nuclear genome, mitochondrial genome and chloroplast genome, and is often used to study the mechanisms of photosynthesis. With the establishment of genetic transformation technology for the genomes of Chlamydomonas reinhardtii, it is also widely used as a cell factory to produce recombinant proteins including vaccines, antibodies, drugs, and so on. As a type of microalgae, Chlamydomonas reinhardtii is widely used in the production of biodiesel due to its high carbohydrate and lipid contents, which has broad application prospects. However, the genetic manipulation system of Chlamydomonas reinhardtii is not yet fully mature. Based on existing research results, from the perspective of production of recombinant proteins, the presence of position effect and gene silencing in the nuclear genetic system can lead to lower production of recombinant proteins, thereby limiting the application of the nuclear genetic system. Relative to cell nucleus, the chloroplast genome is prokaryotic and relatively simple, thus chloroplast become the main research target for production of recombinant proteins. An artificial chloroplast genome of Chlamydomonas reinhardtii has a length of 205 kb, exists in a closed loop manner, contains a total of 99 genes, mainly including genes involved in photosynthesis, transcription, tRNA and rRNA, and the like. In addition to two large-segment inverted repetitive sequences of approximately 22 kb, there are still over 20% of short repetitive sequences distributed between genes in a scattered manner. In addition, the AT content in the chloroplast genome sequence is as high as 65%.

Conventional genetic engineering techniques are merely limited to allowing modifications to existing sequences. Therefore, it will be very meaningful to have the ability to significantly alter and arrange genetic content beyond conventional technical abilities. Therefore, there is a demand for synthetic genomes. Synthetic genomics is the total chemical de novo synthesis of the whole genome or most genomes through a series of technical means. Genome sequences such as yeast alanine tRNA gene and poliovirus, φX174 phage, T7 phage, SARS-like coronavirus and West Nile fever virus have been artificially modified and synthesized successively. The most representative work is the synthesis and minimization of Mycoplasma mycoides genomes, the recoding of Escherichia coli genomes and the artificial synthesis of Saccharomyces cerevisiae chromosomes. The total chemical de novo of a plastid genome is limited to the complete assembly of the rice chloroplast genome by chemically synthesized segments in Bacillus subtilis, but function analysis of the synthesized rice chloroplast genome has not been conducted. In 2012, a Chlamydomonas reinhardtii chloroplast genome was re-assembled in yeast through large segments of the BAC library derived from Chlamydomonas reinhardtii chloroplast genome, achieving synchronous modification of multiple sites within the Chlamydomonas chloroplast genome.

However, so far, the de novo design and in vitro assembly of the chloroplast genome and the functionalization of chloroplast genome synthesis in algae cells have not been achieved. Therefore, the prior art still needs improvement. cl SUMMARY

In view of the defects of the prior art, the present invention provides a method for synthesis, assembly and function test of an artificial chloroplast genome of Chlamydomonas reinhardtii. By using totally chemically synthesized artificial chloroplast genome segments, total chemical de novo synthesis and assembly of a chloroplast genome are achieved in a yeast-bacterium system, which provides a new solution for the rational design, modification and reconstruction of a photosynthetic system of photosynthetic organisms, improvement of photosynthetic efficiency of crops, and solving of agricultural crisis such as food security.

The present invention has the following technical solution:

Provided is a method for synthesis, assembly and function test of an artificial chloroplast genome of Chlamydomonas reinhardtii, the method including de novo design, total chemical synthesis, assembly and function verification of an artificial chloroplast genome of Chlamydomonas reinhardtii, wherein a nucleotide sequence of the artificial chloroplast genome of Chlamydomonas reinhardtii is obtained by designing and modifying a nucleotide sequence of a wild-type Chlamydomonas reinhardtii chloroplast genome and adding a BAC vector backbone, a streptomycin resistance gene aadA, a paromomycin resistance gene aph VIII and an HA tag.

According to the method for synthesis, assembly and function test of an artificial chloroplast genome of Chlamydomonas reinhardtii, the nucleotide sequence of the artificial chloroplast genome of Chlamydomonas reinhardtii has a full length of 221,372 bp.

According to the method for synthesis, assembly and function test of an artificial chloroplast genome of Chlamydomonas reinhardtii, the artificial chloroplast genome of Chlamydomonas reinhardtii is divided into 44 primary segments after being de novo designed, each of the primary segments have 120 bp homologous recombination sequences at two ends.

According to the method for synthesis, assembly and function test of an artificial chloroplast genome of Chlamydomonas reinhardtii, the primary segments are all synthesized through a chemical method.

According to the method for synthesis, assembly and function test of an artificial chloroplast genome of Chlamydomonas reinhardtii, the BAC vector backbone has a length of 11,060 bp, and an insertion site is located at 205,535 bp of the wild-type Chlamydomonas reinhardtii chloroplast genome; the streptomycin resistance gene aadA has a length of 1,630 bp, and an insertion site is located between 173,174 bp and 173,175 bp of the wild-type Chlamydomonas reinhardtii chloroplast genome; the streptomycin resistance gene aadA has a length of 2,287 bp, and an insertion site is located between 71,064 bp and 71,065 bp of the wild-type Chlamydomonas reinhardtii chloroplast genome; a 3×HA-1 tag is located behind atpI, and an insertion site is located between 170,786 bp and 170,787 bp of the wild-type Chlamydomonas reinhardtii chloroplast genome; a 3×HA-2 tag is located behind rps4, and an insertion site is located between 33,292bp and 33293bp of the wild-type Chlamydomonas reinhardtii chloroplast genome.

According to the method for synthesis, assembly and function test of an artificial chloroplast genome of Chlamydomonas reinhardtii, the synthesis and assembly of the artificial chloroplast genome of Chlamydomonas reinhardtii includes the following steps:

6.1 connecting the 44 primary segments of the de novo designed artificial chloroplast genome of Chlamydomonas reinhardtii to the pUC18 vector, respectively;

6.2 co-transforming a BAC vector segment, a screening marker segment, a 7-35 bridging segment, a seg44 segment, segments seg35-seg43, a seg1 segment, and segments seg3-seg7 into yeast strain BY4741, and screening by using the screening medium SC-URA to obtain the yeast strain-1 containing an intermediate plasmid 1;

6.3 co-transforming a pRS415 vector segment and segments seg7-seg21 into yeast strain BY4742, screening by using a screening medium SC-LEU, using a kanMX segment to replace the Met gene on a genome with a yeast strain obtained by screening for Met knockout, and using an SC-LEU+G418 medium for screening to obtain a yeast strain-2 containing an intermediate plasmid 2;

6.4 co-transforming a pRS411 vector segment and segments seg22-seg35 into yeast strain BY4741, and using a screening medium SC-MET for screening to obtain a yeast strain-3 containing an intermediate plasmid 3;

6.5 hybridizing the yeast strain-2 and the yeast strain-3, and screening through an SC-LEU-MET plate; then carrying out spore production and spore division, and screening through an SC-LEU plate and an SC-MET plate; then hybridizing with a yeast SZU-JDY19 and a yeast SZU-JDY20; then hybridizing with the yeast strain-1 containing the intermediate plasmid 1, and screening through an SC-LEU-MET-URA plate; transforming an SZU-ZLP012 plasmid of a strain obtained by screening, and screening through an SC-URA-LEU-MET-HIS plate;

6.6 inducing I-SceI gene expression in the SZU-ZLP012 plasmid by using a galactose, cleaving an I-SceI site to linearize 3 intermediate plasmids, and then obtaining a yeast strain containing a complete artificial chloroplast genome of Chlamydomonas reinhardtii through homologous recombination in a yeast cell.

According to the method for synthesis, assembly and function test of an artificial chloroplast genome of Chlamydomonas reinhardtii, a nucleotide sequence of the intermediate plasmid 1 has a full length of 92,477 bp and contains nucleotide sequences of 1-33,292 bp and 159,554 bp-205,535 bp of the wild-type Chlamydomonas reinhardtii chloroplast genome, and a BAC backbone sequence is added at 205,535 bp of the wild-type Chlamydomonas reinhardtii chloroplast genome; a nucleotide sequence of the intermediate plasmid 2 has a full length of 81,426 bp and contains nucleotide sequences of 28,513 bp-102,566 bp of the wild-type Chlamydomonas reinhardtii chloroplast genome, and pRS406 vector sequences are connected at head and tail interfaces of genome nucleotide sequences; a nucleotide sequence of the intermediate plasmid 3 has a full length of 72,377 bp and contains nucleotide sequences of 97,668 bp-164,450 bp of the wild-type Chlamydomonas reinhardtii chloroplast genome, and pRS411 vector sequences are connected at head and tail interfaces of genome nucleotide sequences.

According to the method for synthesis, assembly and function test of an artificial chloroplast genome of Chlamydomonas reinhardtii, the function test of the artificial chloroplast genome of Chlamydomonas reinhardtii includes the following steps:

8.1 transferring the artificial chloroplast genome of Chlamydomonas reinhardtii into chloroplast of Chlamydomonas reinhardtii CC5168 by particle bombardment, and obtaining positive transformants by streptomycin screening, PCR and Southern Blot screening;

8.2 carrying out homogenization screening on the positive transformants by using gradient streptomycin resistance concentration;

8.3 carrying out Western Blot experiment on the positive transformants for verification of protein expression; and detecting the growth curve of and photosynthesis of the positive transformants.

According to the method for synthesis, assembly and function test of an artificial chloroplast genome of Chlamydomonas reinhardtii, detection segments for screening of the positive transformants are aph VIII, BAC-seg44, and BAC-seg1, respectively.

According to the method for synthesis, assembly and function test of an artificial chloroplast genome of Chlamydomonas reinhardtii, a probe used for Southern Blot verification of the positive transformants is obtained by amplification of aph VIII-F1/R1 and psaA-F/R primers, nucleotide sequences of the aph VIII-F1/R1 primer being shown in SEQ ID NO.1 and SEQ ID NO.2, and nucleotide sequences of the psaA-F/R primer being shown in SEQ ID NO.3 and SEQ ID NO.4.

Beneficial effects are as follows: the present invention provides a method for synthesis, assembly and function test of an artificial chloroplast genome of Chlamydomonas reinhardtii. According to the present invention, rational design has been carried out on the Chlamydomonas reinhardtii chloroplast genome for the first time, and total artificial synthesis of the Chlamydomonas reinhardtii chloroplast genome is proposed. By using the method of synthetic biology, all nucleic acid segments are constructed from chemically synthesized nucleic acid sequences. By using totally chemically synthesized chloroplast genome segments, total chemical de novo synthesis and assembly of a chloroplast genome are achieved in a yeast-bacterium system. Then, a totally chemically synthesized chloroplast genome is transformed into Chlamydomonas cells to replace the original chloroplast genome through various technical means, which works normally, and has been verified, fulfilling biological functions of the totally chemically synthesized chloroplast genome. The method can be widely applied in gene editing of the Chlamydomonas reinhardtii chloroplast genome and production of antibody drugs and the like, and has huge commercial advantages and broad market prospects. Meanwhile, according to the present invention, the Chlamydomonas reinhardtii chloroplast genome is an efficient platform for carrying out synthetic biology operation, the de novo design, total chemical synthesis, in vitro assembly and identification of the genome thereof provide a new solution for the rational design, modification and reconstruction of a photosynthetic system of photosynthetic organisms, improvement of photosynthetic efficiency of crops, and solving of agricultural crisis such as food security.

### BRIEF DESCRIPTION OF THE DRAWINGS

FIG. 1 is a map of an artificial chloroplast genome of Chlamydomonas reinhardtii provided by an embodiment of the present invention.

FIG. 2 is a map of an intermediate plasmid 1 provided by an embodiment of the present invention.

FIG. 3 is a map of an intermediate plasmid 2 provided by an embodiment of the present invention.

FIG. 4 is a map of an intermediate plasmid 3 provided by an embodiment of the present invention.

FIG. 5 is a schematic diagram of Junction PCR results of the intermediate plasmid 1 provided by an embodiment of the present invention.

FIG. 6 is a schematic diagram of Junction PCR results of the intermediate plasmid 2 provided by an embodiment of the present invention.

FIG. 7 is a schematic diagram of Junction PCR results of the intermediate plasmid 3 provided by an embodiment of the present invention.

FIG. 8 is a schematic diagram of hybridization processes of yeasts containing the intermediate plasmid 1, the intermediate plasmid 2 and the intermediate plasmid 3 provided by an embodiment of the present invention.

FIG. 9 is a schematic diagram of total Junction PCR verification results of the artificial chloroplast genome of Chlamydomonas reinhardtii provided by an embodiment of the present invention.

FIG. 10 is a schematic diagram of positive screening results of transformants of the artificial chloroplast genome of Chlamydomonas reinhardtii provided by an embodiment of the present invention.

FIG. 11 is a schematic diagram of Southern Blot experiment results provided by an embodiment of the present invention.

FIG. 12 is a schematic diagram of culture results of transformants on TAP mediums with different streptomycin concentrations provided by an embodiment of the present invention.

FIG. 13 is a schematic diagram of Western Blot experiment results provided by an embodiment of the present invention.

FIG. 14 is a schematic diagram of growth status results of CC5168 synthetic chloroplast algae strains provided by an embodiment of the present invention.

FIG. 15 is a schematic diagram of photosynthetic efficiency results of CC5168 transformants provided by an embodiment of the present invention.

FIG. 16 is a schematic diagram of results of CC5168 synthetic chloroplast algae strains restoring normal growth under light provided by an embodiment of the present invention.

### DETAILED DESCRIPTION OF THE EMBODIMENTS

The present invention provides a method for synthesis, assembly and function test of an artificial chloroplast genome of Chlamydomonas reinhardtii. In order to make the purpose, technical solution, and effects of the present invention clearer and more explicit, the present invention is further illustrated in detail below. It should be understood that the specific embodiments described herein are only used to explain the present invention, but not to limit the present invention.

An embodiment of the present invention provides a method for synthesis, assembly and function test of an artificial chloroplast genome of Chlamydomonas reinhardtii, the method including de novo design, total chemical synthesis, assembly and function verification of an artificial chloroplast genome of Chlamydomonas reinhardtii. According to the embodiment of the present invention, by adopting the method of synthetic biology, by using totally chemically synthesized chloroplast genome segments, total chemical de novo synthesis and assembly of a chloroplast genome are achieved in a yeast-bacterium system. Then, a totally chemically synthesized chloroplast genome is transformed into Chlamydomonas cells to replace the original chloroplast genome through various technical means, fulfilling biological functions of the totally chemically synthesized chloroplast genome.

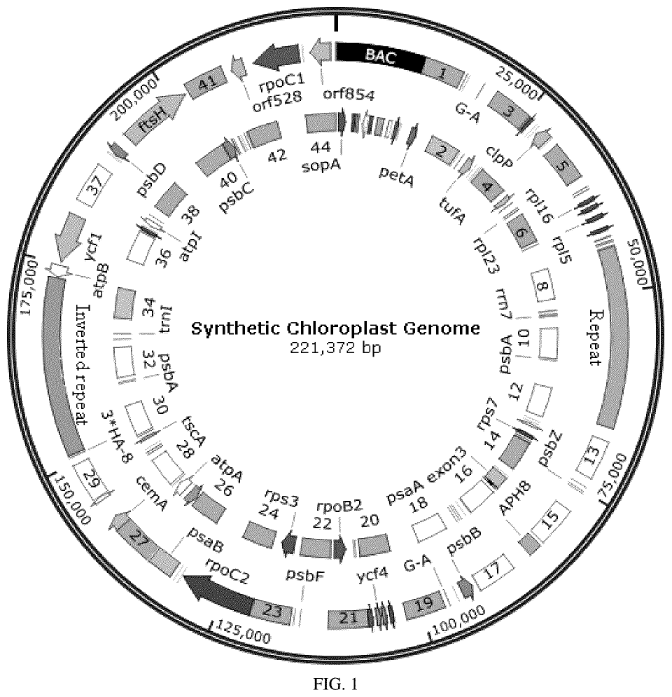

In some implementations, a nucleotide sequence of the artificial chloroplast genome of Chlamydomonas reinhardtii is obtained by designing and modifying a nucleotide sequence of a wild-type Chlamydomonas reinhardtii chloroplast genome and adding a BAC vector backbone, a streptomycin resistance gene aadA, a paromomycin resistance gene aph VIII and an HA tag. The finally designed and obtained artificial chloroplast genome of Chlamydomonas reinhardtii is shown in FIG. 1.

In some implementations, the nucleotide sequence of the artificial chloroplast genome of Chlamydomonas reinhardtii has a total of 221.372 bp bases.

In some implementations, the nucleotide sequence of the wild-type Chlamydomonas reinhardtii chloroplast genome has a full length of 205,535 bp, with NCBI Sequence Number of NC_005353.1.

In some implementations, the artificial chloroplast genome of Chlamydomonas reinhardtii is divided into 44 primary segments when being de novo designed, each of the primary segments having 120 bp homologous recombination sequences at two ends. Distribution of the 44 primary segments is shown in FIG. 1.

In some implementations, the primary segments are all synthesized through a chemical method. All the primary segments are connected to a pUC18 vector. Meanwhile, the primary segments can also be obtained by digestion on the vector by a restriction enzyme Not I.

In some implementations, the BAC vector backbone, aphVIII and aadA resistance screening markers, and the HA tag are added to the artificial chloroplast genome of Chlamydomonas reinhardtii.

In some implementations, the BAC vector backbone has a length of 11,060 bp, and an insertion site is located at 205,535 bp of the wild-type Chlamydomonas reinhardtii chloroplast genome; the streptomycin resistance gene aadA has a length of 1,630 bp, and an insertion site is located between 173,174 bp and 173,175 bp of the wild-type Chlamydomonas reinhardtii chloroplast genome; the streptomycin resistance gene aadA has a length of 2,287 bp, and an insertion site is located between 71,064 bp and 71,065 bp of the wild-type Chlamydomonas reinhardtii chloroplast genome; a 3×HA-1 tag is located behind atpI, and an insertion site is located between 170,786 bp and 170,787 bp of the wild-type Chlamydomonas reinhardtii chloroplast genome; a 3×HA-2 tag is located behind rps4, and an insertion site is located between 33,292 bp and 33293 bp of the wild-type Chlamydomonas reinhardtii chloroplast genome.

In some implementations, the synthesis and assembly of the artificial chloroplast genome of Chlamydomonas reinhardtii includes the following steps:

S01, connecting the 44 primary segments of the de novo designed artificial chloroplast genome of Chlamydomonas reinhardtii to a pUC18 vector, respectively;

S02, co-transforming a BAC vector segment, a screening marker segment, a 7-35 bridging segment, a seg44 segment, segments seg35-seg43, a seg1 segment, and segments seg3-seg7 into a yeast BY4741, and screening by using a screening medium SC-URA to obtain a yeast strain-1 containing an intermediate plasmid 1;

S03, co-transforming a pRS415 vector segment and segments seg7-seg21 into a yeast BY4742, screening by using a screening medium SC-LEU, using a kanMX segment to replace a Met gene on a genome with a yeast strain obtained by screening for Met knockout, and using an SC-LEU+G418 medium for screening to obtain a yeast strain-2 containing an intermediate plasmid 2;

S04, co-transforming a pRS411 vector segment and segments seg22-seg35 into a yeast BY4741, and using a screening medium SC-MET for screening to obtain a yeast strain -3 containing an intermediate plasmid 3;

S05, hybridizing the yeast strain-2 and the yeast strain-3, and screening through an SC-LEU-MET plate; then carrying out spore production and spore division, and screening through an SC-LEU plate and an SC-MET plate; then hybridizing with SZU-JDY19 (Preservation Number: CCTCC M 20221034) and SZU-JDY20 (Preservation Number: CCTCC M 20221033); then hybridizing with the yeast strain-1 containing the intermediate plasmid 1, and screening through an SC-LEU-MET-URA plate; transforming an SZU-ZLP012 plasmid (Preservation Number: CCTCC M 20221031) of a strain obtained by screening, and screening through an SC-URA-LEU-MET-HIS plate;

S06, inducing I-SceI gene expression in the SZU-ZLP012 plasmid by using galactose, cleaving an I-SceI site to linearize 3 intermediate plasmids, and then obtaining a yeast strain (Preservation Number: CCTCC M 20221035) containing a complete artificial chloroplast genome of Chlamydomonas reinhardtii through homologous recombination in a yeast cell.

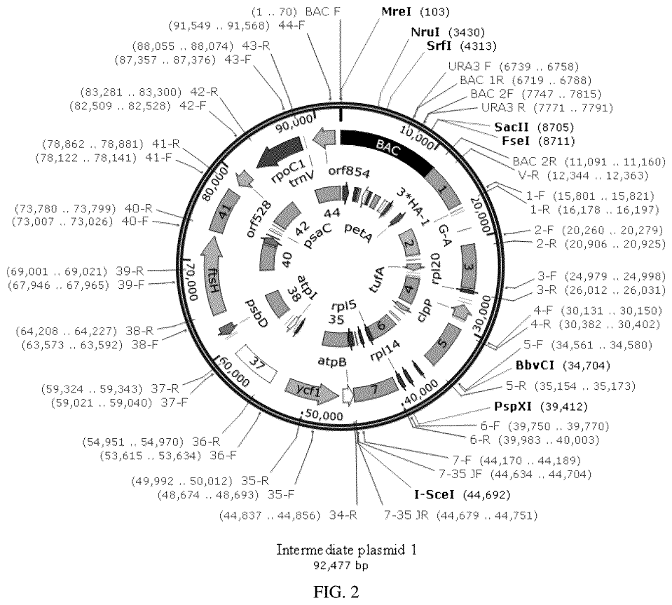

In some implementations, a nucleotide sequence of the intermediate plasmid 1 has a full length of 92,477 bp and contains nucleotide sequences of 1-33,292 bp and 159,554 bp-205,535 bp of the wild-type Chlamydomonas reinhardtii chloroplast genome, and a map thereof is shown in FIG. 2. A BAC backbone sequence is added at 205,535 bp of the wild-type Chlamydomonas reinhardtii chloroplast genome, and is shown in SEQ ID NO.5.

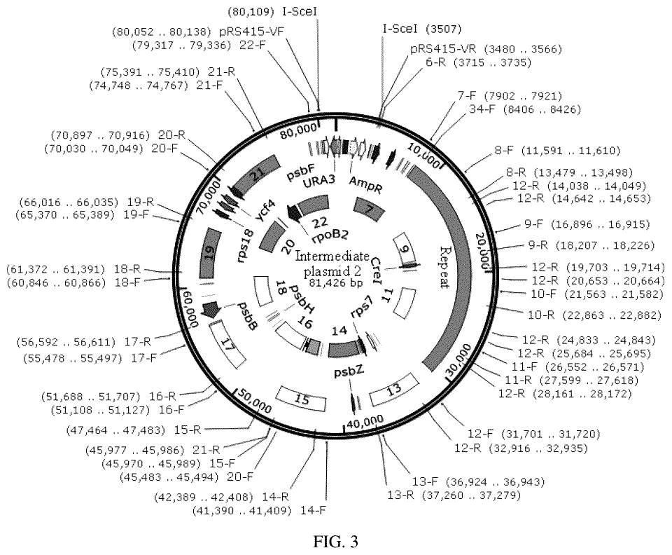

In some implementations, a nucleotide sequence of the intermediate plasmid 2 has a full length of 81,426 bp and contains nucleotide sequences of 28,513 bp-102,566 bp of the wild-type Chlamydomonas reinhardtii chloroplast genome, pRS406 vector sequences are connected at head and tail interfaces of genome nucleotide sequences, and a map thereof is shown in FIG. 3; the pRS406 vector sequences are shown in SEQ ID NO.6.

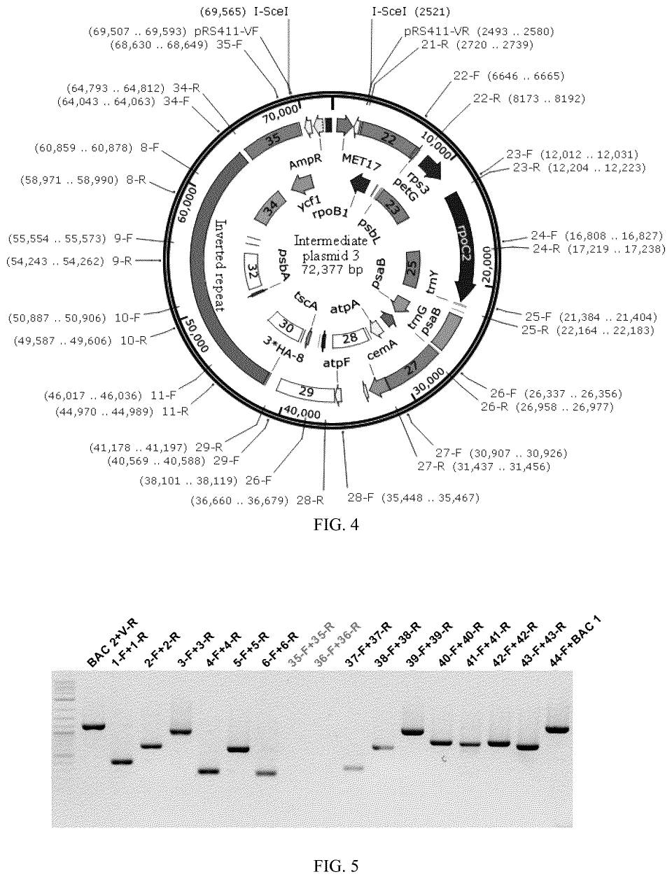

In some implementations, a nucleotide sequence of the intermediate plasmid 3has a full length of 72,377 bp and contains nucleotide sequences of 97,668 bp-164,450 bp of the wild-type chloroplast genome, pRS411 vector sequences are connected at head and tail interfaces of genome nucleotide sequences, and a map thereof is shown in FIG. 4; the pRS411 vector sequence is shown in SEQ ID NO.7.

In some implementations, in step S03, a kanMX segment is used to replace a Met gene on a genome with a yeast strain obtained by screening for Met knockout; pFA6-kanMX4 (commercial plasmid, purchased from Shanghai Yaji Biotechnology Co., Ltd., Article Number: YC-14391RJ) is used as a template for PCR amplification by using a primer Met17F/R to obtain a kanMX segment, the pFA6-kanMX4 being purchased from a commercial company; PCR segments of kanMX are transformed into a yeast containing the intermediate plasmid 2, subjected to screening culture, and screened with SC-LEU+G418; a transformed plate is photocopied onto a plate of G418, and monoclones that can grow on both plates are selected, that is, screening is carried out to obtain the yeast strain-2.

Specifically, a sequence of the kanMX segment is shown in SEQ ID NO.8; sequences of primers Met17-F and Met17-R are shown in SEQ ID NO.9 and SEQ ID NO.10.

Specifically, a ZLP012 plasmid carries I-SceI endonuclease and HIS3auxotrophic screening marker genes. The expression of I-SceI is induced by using the galactose, and chunk1 (intermediate plasmid 1), chunk2 (intermediate plasmid 2) and chunk3 (intermediate plasmid 3) linearize three intermediate plasmids in a yeast due to the cleavage of the I-SceI site.

According to the embodiment of the present invention, when the artificial chloroplast genome of Chlamydomonas reinhardtii is assembled, firstly, a vector segment is amplified by PCR to carry homologous sequences with both ends of segments; afterwards, the carrier segment and corresponding segments are co-transformed into yeast cells; then, screening is carried out by an appropriate auxotrophic SC screening medium to obtain transformants, genomes of yeast transformants are extracted, and correctly assembled chunk1, chunk2, and chunk3 are obtained by Junction PCR screening. An I-SceI endonuclease recognition site is added at each intermediate plasmid. After all secondary segments are introduced into a yeast, the intermediate plasmids are linearized by inducing the expression of I-SceI endonuclease in cells, and the three secondary segments are assembled into a final artificial synthetic chloroplast genome by using a homologous recombination mechanism.

In some implementations, the function test of the artificial chloroplast genome of Chlamydomonas reinhardtii includes the following steps:

S100, transferring the artificial chloroplast genome of Chlamydomonas reinhardtii into a chloroplast of Chlamydomonas reinhardtii CC5168 by particle bombardment, and obtaining positive transformants by streptomycin screening, PCR and Southern Blot screening;

S200, carrying out homogenization screening on the positive transformants by using gradient streptomycin resistance concentration;

S300, carrying out Western Blot experiment on the positive transformants for verification of protein expression; and detecting a growth curve of the positive transformants and photosynthesis of a repair mutant strain.

Specifically, a concentration of a streptomycin screening medium is 150 μg/mL. The homogenized gradient streptomycin concentrations are 150 μg/mL, 300 μg/mL, 400 μg/mL, 500 μg/mL, 600 μg/mL, 700 μg/mL, 800 μg/mL, 900 μg/mL and 1000 μg/mL.

During function analysis of the artificial chloroplast genome of Chlamydomonas reinhardtii, firstly, a yeast plasmid is extracted, and transferred into Escherichia coli EPI300 by electroporation, an Escherichia coli plasmid is extracted, a synthetic chloroplast genome is transferred into photo-deficient Chlamydomonas reinhardtii CC5168 (ΔpsbH) by biolistic transformation, and transformants are obtained through antibiotic screening and HS medium screening, PCR and Southern Blot positive screening are carried out on the transformants; Finally, Western Blot verification is carried out on the positive transformants, and meanwhile, the growth curve of the transformants and photosynthesis repair of photo-deficient algal strains are detected.

In some implementations, detection segments for identification and positive screening of the positive transformants are aph VIII, BAC-seg44, and BAC-seg1, respectively, and detection primers are aphVIII-F/R, BAC44-F/R, and BAC1-F/R, respectively; nucleotide sequences of the aphVIII-F/R are shown in SEQ ID NO.11 and SEQ ID NO.12, nucleotide sequences of the BAC44-F/R are shown in SEQ ID NO.13 and SEQ ID NO.14, and nucleotide sequences of the BAC1-F/R are shown in SEQ ID NO.15 and SEQ ID NO.16.

In some implementations, probes used for Southern Blot verification of the positive transformants are obtained by amplification of aph VIII-F1/R1 and psaA-F/R primers, nucleotide sequences of the aph VIII-F1/R1 primer are shown in SEQ ID NO.1 and SEQ ID NO.2, and nucleotide sequences of the psaA-F/R primer are shown in SEQ ID NO.3 and SEQ ID NO.4.

The embodiment of the present invention provides implementations and methods for the design, synthesis, assembly, and expression of synthetic genomes. Methods are included for: rational design of genomes; preparation of small nucleic acid segments and assembly of them into expression cassettes containing genomic components; correction of errors in expression cassette sequences; assembly of the expression cassettes into synthetic genomes (for example, through in vitro recombination); screening and verification of transferring of synthetic genomes into Chlamydomonas reinhardtii chloroplasts and positive transformed algae strains by using chloroplasts. The present invention includes methods for the rational design of synthetic genomes and construction of synthetic genomes, including preparation and assembly of genomic nucleic acid components, wherein all genomes are constructed from chemically synthesized nucleic acid components. In a specific implementation, a complete synthetic genome is constructed entirely from chemically synthesized nucleic acid components or copies of the chemically synthesized nucleic acid components. Further, the synthetic genome can be a synthetic organelle genome.

According to the present invention, by adopting the method of synthetic biology, by using totally chemically synthesized chloroplast genome segments, total chemical de novo synthesis and assembly of a chloroplast genome are achieved in a yeast-bacterium system. Then, a totally chemically synthesized chloroplast genome is transformed into Chlamydomonas cells to replace the original chloroplast genome through various technical means, fulfilling biological functions of the totally chemically synthesized chloroplast genome. According to the present invention, the Chlamydomonas reinhardtii chloroplast genome is an efficient platform for carrying out synthetic biology operation, the de novo design, total chemical synthesis, in vitro assembly and identification of the genome thereof provide a new solution for the rational design, modification and reconstruction of a photosynthetic system of photosynthetic organisms, improvement of photosynthetic efficiency of crops, and solving of agricultural crisis such as food security.

The method for synthesis, assembly and function test of an artificial chloroplast genome of Chlamydomonas reinhardtii of the present invention is further explained and illustrated below through specific embodiments.

### Embodiment 1 Design of Artificial Chloroplast Genome of Chlamydomonas reinhardtii

The sequence (NC_005353.1) of a wild-type Chlamydomonas reinhardtii chloroplast genome was downloaded from NCBI, and designed based on a sequence of a wild-type chloroplast genome, including insertion of an HA tag to detect expression of a target protein, and insertion of two resistance gene expression boxes, 5′-atpA-aadA-rbcL-3′ and 5′atpA-aph VIII-rbcL-3′, for subsequent screening. The 5′atpA-aph VIII-rbcL-3′ had a resistance screening marker length of 2,287 bp, located between 71,064 bp and 71,065 bp of the wild-type Chlamydomonas reinhardtii chloroplast genome. The aadA had a resistance screening marker length of 1,630 bp, located between 173,174 bp and 173,175 bp of the wild-type Chlamydomonas reinhardtii chloroplast genome. A 3×HA-1 tag was behind atpI, and between 170,786 bp and 170,787 bp. A 3×HA-2 tag was behind rps4, and between 33,292 bp and 33293 bp. Through de novo design, the sequence of the artificial chloroplast genome of Chlamydomonas reinhardtii with a full length of 221,372 bp was obtained, as shown in FIG. 1.

### Embodiment 2 Assembly of Artificial Chloroplast Genome of Chlamydomonas reinhardtii

A commercial company was commissioned to synthesize all primary segments. All the primary segments were connected to a pUC18 vector and could be obtained by a restriction enzyme Not I. The above sequences were confirmed by sequencing and digestion.

### 1. Assembly of Intermediate Plasmid 1

Assembly was carried out in a BY4741 background strain (commercial strain, purchased from Huinuo Biomedical Technology Co., Ltd., Article Number: A226), with a total of 21 segments, including 17 chloroplast genome synthetic segments (including nucleotide sequences of 0-33,292 bp and 159,554 bp-205,535 bp of a wild-type chloroplast genome), 2 BAC segments, a URA3 screening gene, and a 7-35 bridging segment. BAC served as a vector for an intermediate plasmid 1, the URA3 gene provided an auxotrophic screening marker, and the 7-35 bridging segment was used for insertion of an I-SceI digestion site between segment 7 and segment 35. A map of the finally obtained intermediate plasmid 1 is shown in FIG. 2. A specific assembly process was as follows:
1) Segment preparation: synthetic segments 1-7 and 35-44 were obtained through Not I digestion, and by using a pRS416 plasmid (commercial plasmid, purchased from ACD, Article Number: 518681-C2) as a template, and using vecF+vecR as primers, through PCR amplification, a pRS416 vector segment with homologous sequences of segment a and segment d was obtained. The pRS416 amplified segment, seg-a, seg-b1, seg-b2, seg-c, and seg-d segments were co-transformed into a BY4741 yeast (commercial strain, purchased from Huinuo Biomedical Technology Co., Ltd., Article Number: A226), and screening was carried out by an SC-URA screening medium to obtain segment 2. By using pJS356 (pJS356 Cloned pTERM+415, DNA) as a template and using BAC F/1R and BAC 2F/2R as primers, through PCR amplification, BAC vector segments were obtained. By using pRS406 (publicly available plasmid, see Sikorski and Hieter, 1989) (pRS406-ua3, DNA) as a template and using URA3 F and URA3 R as primers, through PCR amplification, screening marker segments were obtained. 7-35 segments were obtained through 7-35 JF/JR Primer anealing, and segment seg44 was obtained through 44-V L/R PCR.
2) Yeast transformation: vector segments, seg35-seg43, seg1, and seg3-seg7 were co-transformed into a yeast BY4741, and screening was carried out by using a screening medium SC-URA to obtain transformants.
3) Identification: a toothpick was used to pick the yeast transformants into sterilized ddH2O, and even vortex mixing was carried out, with 4 cycles at 98° C., 3 min, 4° C., 2 min. Centrifugation was carried out at 6000 rpm for 5 min. By using supernatant as a template and using 6F/R and 41F/R as primers, PCR amplification was carried out, and the transformants were subjected to positive primary screening. A genome of yeast strains that were primarily screened as positive was extracted. Junction PCR verification was carried out by using a total of 18 pairs of primers, i.e., BAC2/V-R, 1F/1R to 6F/6R, 7F/7-35 JR, 35F/35R to 43F/43R and 44F/BAC1. Verification results are shown in FIG. 5, indicating that the intermediate plasmid 1 is successfully assembled in BY4741 cells. A map of the intermediate plasmid 1 is shown in FIG. 2.

### 2. Assembly of Intermediate Plasmid 2

1) Segment preparation: synthetic segments seg7-seg21 were obtained through

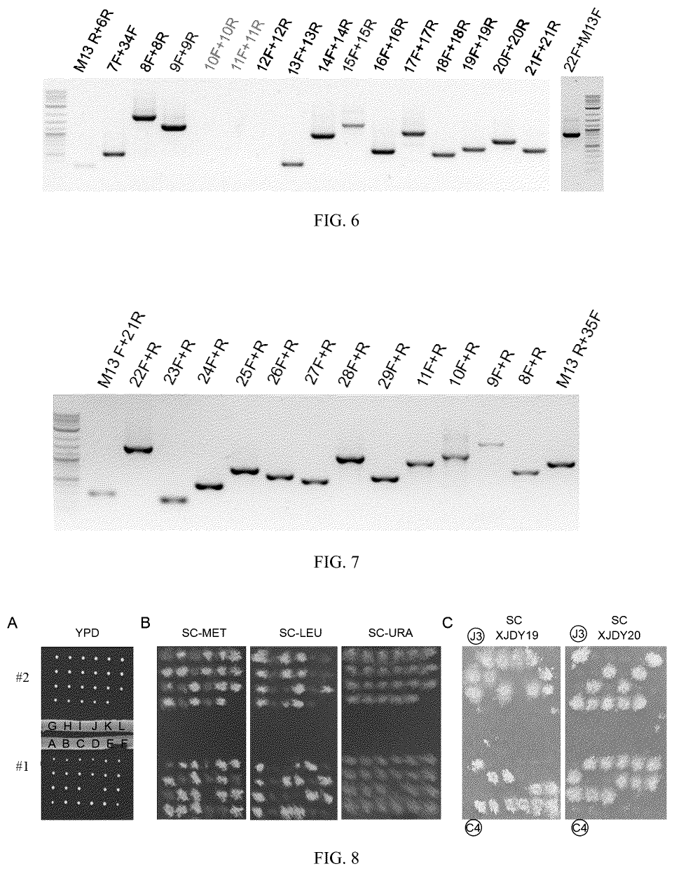

Not I digestion, and by using pRS415 (publicly available plasmid, see Sikorski and Hieter, 1989) (-Leu) as a template, a pRS415 vector segment with homologous segments with seg7 and seg22 was amplified.
2) Yeast transformation: vector segments and segments seg7-seg21 were co-transformed into a yeast BY4742 (commercial strain, purchased from ZOMANBIO, Article Number: ZK280), and screening was carried out by using a screening medium SC-LEU to obtain transformants.
3) Positive identification: a positive identification process was the same as that of an intermediate plasmid 2, and transformant positive primary screening primers were 7F/34F and 18F/R. Junction PCR verification was carried out by using a total of 14 pairs of primers, i.e., 7F/34F and 8F/8R to 20F/20R. Verification results are shown in FIG. 6, and the research results indicate that the intermediate plasmid 2 is successfully assembled in the yeast BY4742. A map of the intermediate plasmid 2 is shown in FIG. 3.
4) By using pFA6-kanMX4 (commercial plasmid, purchased from Shanghai Yaji Biotechnology Co., Ltd., Article Number: YC-14391RJ) as a template, through primers Met17F/R, PCR amplification was carried out to obtain a kanMX segment. The kanMXPCR segment was transformed into a yeast containing the intermediate plasmid 2, screening culture, and screening with SC-LEU+G418 were carried out; a transformed plate was photocopied onto a plate of G418, and monoclones that could grow on both plates were selected. Screening was carried out to obtain a yeast strain-2.

### 3. Assembly of Intermediate Plasmid 3

1) Segment preparation: synthetic segments seg22-seg35 were obtained through Not I digestion, and by using pRS411 (-Met) (publicly available plasmid, see Sikorski and Hieter, 1989) as a template, through primers pRS411VF+VR, PCR amplification was carried out to obtain a segment with homologous sequences with seg22 and seg35.
2) Yeast transformation: vector segments and segments seg22-seg35 were co-transformed into a yeast BY4741, and screening was carried out by using a screening medium SC-MET to obtain transformants.
3) Positive identification: a yeast transformant screening process was the same as that of an intermediate plasmid 3. Primary screening primers for transformant positive primary screening were 26F/R and 34F/R. Junction PCR verification was carried out on strains that were primarily screened positive by using a total of 13 pairs of primers, i.e., 22F/R to 29F/R, 8F/R to 11F/R and 34F/R. Verification results are shown in FIG. 7, and the research results indicate that the intermediate plasmid chunk3 is successfully assembled in the yeast BY4741. A map of the intermediate plasmid 3 is shown in FIG. 4.

### 4. Assembly of Complete Synthetic Chloroplast Genome

### 1) Obtaining Yeast Strain Haploid Containing Both Intermediate Plasmid 2 and Intermediate Plasmid 3

Diploid yeast strains containing both the intermediate plasmid 2 (LEU2) and the intermediate plasmid 3 (MET17) were screened by using an SC-LEU-MET plate. The diploid yeast strains (#1, #2) were randomly selected from the SC-LEU-MET plate for spore production and spore division to obtain a haploid yeast (as shown in FIG. 8A). A YPD plate was photocopied onto SC-LEU, SC-MET, and SC-URA plates, respectively. The SC-LEU and SC-MET plates were used for screening haploid yeast strains containing both the intermediate plasmid 2 and the intermediate plasmid 3. The SC-URA plate was used for ensuring that an obtained haploid did not grow on the SC-URA plate for subsequent hybridization of three intermediate plasmids. The haploid yeast strains containing both the intermediate plasmid 2 and the intermediate plasmid 3 were obtained (as shown in FIG. 8 B).

### 2) Obtaining Yeast Strain Containing All Intermediate Plasmid 1, Intermediate Plasmid 2 and Intermediate Plasmid 3

In order to subsequently hybridize with intermediate plasmid 1, it was necessary to select haploid yeast strains with a mating type of alpha from the haploid yeast strains. Mating types of SZU-JDY19 (MAT a thr4 Mal′) (Preservation Number: CCTCC M 20221034) and SZU-JDY20 (MAT alpha thr4 Mal′) (Preservation Number: CCTCC M 20221033) were a and alpha, respectively, and a haploid strain that was hybridized with them could grow on an SD plate only after successfully hybridized into a diploid. The YPD spore division plates mentioned above were hybridized with SZU-JDY19 and SZU-JDY20, and then photocopied onto an SD plate to successfully screen a target strain, which was then, hybridized with a yeast strain containing the intermediate plasmid 1, and screening was carried out through an SC-LEU-MET-URA plate to obtain a yeast strain containing all three intermediate plasmids (as shown in FIG. 8 C).

3) Assembly of Complete Chlamydomonas reinhardtii Chloroplast Genome by Three Intermediate Plasmids in Yeast Strain

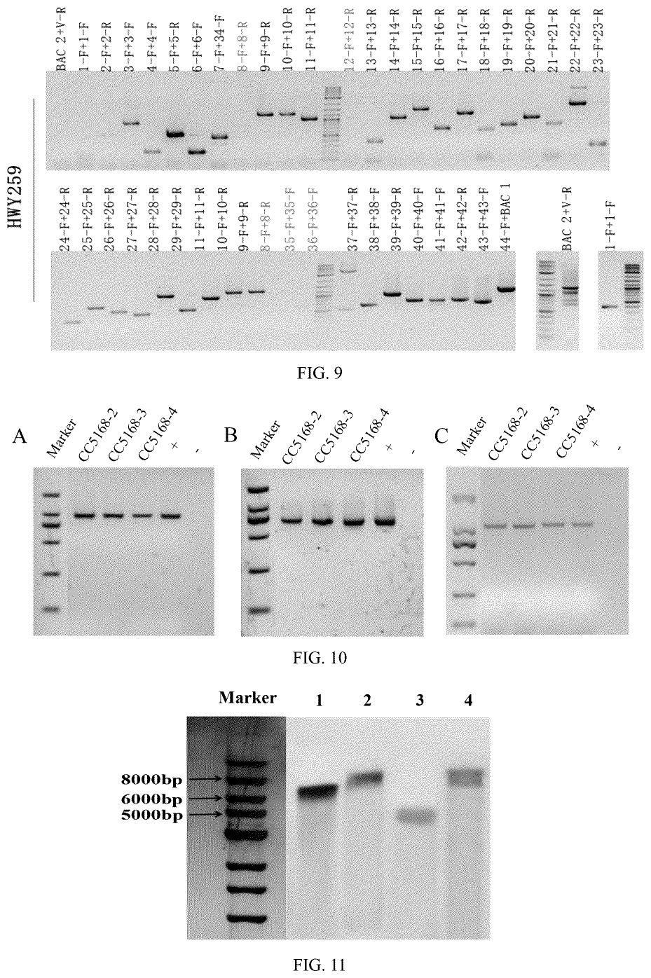

Among the three intermediate plasmids, each pair has a homologous segment. The intermediate plasmid 1 and the intermediate plasmid 2 both have segment 7, the intermediate plasmid 2 and the intermediate plasmid 3 both have segment 22, the intermediate plasmid 3 and the intermediate plasmid 1 both have segment 35, and one end of the homologous segment has an I-SceI cleavage site. I-SceI is an intron-encoded endonuclease of Saccharomyces cerevisiae mitochondria, which can specifically recognize about 18 bp sequences, and generate a double strand break nick at a recognition site to activate a homologous recombination repair mechanism of yeast cells. Transformation of an SZU-ZLP012 plasmid (Preservation Number: CCTCC M 20221031) was carried out. Screening was carried out by an SC-URA-LEU-MET-HIS plate. Expression of an I-SceI gene in a ZLP012 plasmid was induced by using a galactose to linearize the intermediate plasmid 1, the intermediate plasmid 2 and the intermediate plasmid 3. By means of the homologous recombination repair mechanism of yeast cells, assembly of a complete chloroplast genome with a BAC vector of the intermediate plasmid 1 as a vector was achieved. A genome of yeast strains was extracted. PCR screening was carried out by using 44 Junction primers to obtain a strain (Preservation Number: CCTCC M 20221035) containing a complete synthetic artificial chloroplast genome of Chlamydomonas reinhardtii (as shown in FIG. 9).

### Embodiment 4 Function verification of artificial chloroplast genome of Chlamydomonas reinhardtii

1. Transfer of Chloroplast Artificial Genome into Chlamydomonas reinhardtii Chloroplast

### 1) Preparation of Gold Powder

30 mg of gold powder with a diameter of 1 μm was weighed and placed in a 1.5 mL centrifuge tube, 1 mL of 70% ethanol was added, Vigorous shake washing was carried out for 5 min, Soaking was carried out for 15 min. Centrifugation was carried out at 4000 rpm for 5 s, and the supernatant was discarded. 1 mL of ddH2O was added, Vigorous shake vortex was carried out for 1 min. Standing was carried out for 1 min. Repeated washing was carried out 3 times, and centrifugation was carried out at 4000 rpm for 5 min. 500 μL of 50% glycerol was added to a gold powder precipitate, and the gold powder precipitate was stored at 4° C. for standby use.

### 2) Envelope of Gold Powder

The gold powder stored in the 50% glycerol was fully shaken for 5 min, and 50 uL of the gold powder was taken and placed into a 1.5 mL centrifuge tube. 5 μg of a plasmid, 50 μL of CaCl₂ and 20 μL of 0.1 M spermidine were added at a time. Full vortex shaking was carried out for 3 min. Standing was carried out for 1 min. Centrifugation was carried out at 4000 rpm for 5 s. The supernatant was discarded, sufficient 70% ethanol was added for rinsing, and the supernatant was discarded, repeated twice. After rinsing, sufficient anhydrous ethanol was added for rinsing, and the supernatant was discarded. An appropriate amount of anhydrous ethanol was added. Quick vortex was carried out for 5 s. A precipitate was resuspended.

### 3) Biolistic Transformation

A receptor algal cell cultured to a cell concentration of 2×10⁶ cell/mL was centrifuged (3500× g, 5 min) for collection, and resuspended with a fresh TAP medium to 1×10⁸ cell/mL. 250 μL of a resuspended algal cell was taken, laid at a center of a 90×15 mm screening plate, and dried for 2 hours in dark at a room temperature. Preparation for bombardment DNA particles: 50 μL of gold powder was taken, add 5.0 μL of DNA (1.0 μg/μL), 50 μL of 2.5 M CaCl₂, and 20 μL of 0.1 mM spermidine were added in sequence, shaking for mixing well was carried out, centrifugation was carried out, washing was carried out with 70% alcohol, and finally resuspending was carried out with 100 μL of anhydrous ethanol, taking 10 μL for bombardment each time. A gene gun was Biolistic PDS-1000/He Particle Delivery System (BIO-RAD), with the following bombardment parameters: a crackable film 1100 psi, and a bombardment distance of 9 cm. After bombardment was completed, the plates were cultured in dark at 25° C. for 24 hours, and then transferred into an incubator at 25° C. with a photoperiodic ratio of 16:8 and continued to culture for 3-4 weeks until green monoclones grew.

### 2. Identification of Transformants

After green transformants grew, in a super clean bench, the transformants were streaked with a toothpick to a new streptomycin resistance (150 μg/mL) culture dish, and continued to culture. Chlamydomonas reinhardtii monoclone cells were picked up to 50 μL of chelex. After shaking and mixing well at 98° C. for 30 min, immediate cooling was carried out on ice. Shaking for mixing well and centrifugation were carried out. By using a supernatant as a template and using 3×HA-1F/R and 3×HA-2 as primers for PCR amplification, transformant positive primary screening was carried out. Subsequently, a genome of transformants subjected to HA primary screening positive was extracted by using a genome extraction kit. By using the genome as a template, PCR amplification was carried out by using primers such as aph VIII-F/R, BAC44-F/R and BAC1-F/R. PCR products were subjected to 1.2% agarose gel electrophoresis for further positive identification to obtain transformants containing all HA tag, aphVIII and BAC sequences. Results are shown in FIG. 10, where A, aph VIII PCR identification; B, BAC-Seg1 PCR identification; C, BAC-seg44 PCR identification, in abc, Lane-has no template as negative control, Lane+ is plasmid as positive control, Lanes CC125-K3, CC125-K6, CC125-M8, CC125-K2, CC125-N15, CC5168-2, CC5168-3 and CC5168-4 are transformants, and Marker is DL2000 bp.

### 3. Southern Blot Verification

aph VIII and psaA specific amplification primers aphVIII-F1/R1 and psaA-F/R were designed, by using chunk2 as a template, PCR amplification was carried out to obtain aph VIII and psaA probe segments, and aphVIII and psaA probes were marked respectively by using DIG DNA Labeling and Detection Kit. Wild-type algal strain CC5168 (commercial strain purchased from Chlamydomonas Resource Center, USA) and transformant CC5168-3 were cultured to a logarithmic phase and centrifuged to collect algal cells. Hind III and EcoR V were selected to respectively subject 10-20 ug genome DNA to digestion at 37° C. for 6 h, and electrophoresis was carried out for 90 min at 80 V. In a 10×SSC transfer buffer solution, the genome DNA was transferred overnight to a positively charged nylon membrane, and then subjected to ultraviolet crosslinking (1500 V, 1.5 min) for fixation. The fixed nylon membrane was pre-hybridized for 30 min at 28° C. with 28° C. preheated DIG Easy Hyb Buffer. The pre-hybridized solution was discarded. Fresh 28° C. preheated DIG Easy Hyb Buffer (3.5 mL/100 cm²) was added, and hybridization verification was carried out at 28° C. with aph VIII and psaA probes, refer to the instructions for specific operations.

Genome DNA of a photo-deficient algal strain CC5168 and a positive transformed algal strain CC5168-3 was subjected to enzymolysis with EcoR I and then respectively hybridized with the psaA probe. Results are shown in FIG. 11, where Lane 1 represents a hybridization result of enzymatic digestion of a wild-type CC125 genome by the psaA probe and the EcoR I; Lane 2 represents a hybridization result of enzymatic digestion of a CC125-N15 genome by the psaA probe and the EcoR I; Lane 3 represents a hybridization result of enzymatic digestion of the CC125-N15 genome by the aph VIII probe and Hind III; Lane 4 represents a hybridization result of enzymatic digestion of the CC125-N15 genome by the aph VIII probe and the EcoR I. Lane 1 is compared to Lane 2, and Lane 3 is compared to Lane 4. Marker is DL10000 bp. Results show that a hybrid band of Chlamydomonas reinhardtii CC5168 is approximately 6,681 bp, and a hybrid band of CC5168-3 is approximately 9000 bp, indicating that an aph VIII expression cassette exists in an artificially synthesized chloroplast genome. Subsequently, a genome of a positive transformed algal strain CC5168-3 was subjected to enzymolysis with EcoR I and Hind III, and subjected to Southern Blot with an aph VIII probe. Hybridization results show that a hybrid band of CC5168-3 genome DNA subjected to enzymolysis with the aph VIII probe and the EcoR I is approximately 9,000 bp, which is consistent with that with the psaA probe. Meanwhile, a hybrid band of CC5168-3 genome DNA subjected to enzymolysis with an aph VIII probe and Hind III is approximately 5,000 bp, indicating that an aph VIII expression cassette exists between 79,599 bp and 88,566 bp of an artificially synthesized chloroplast genome (FIG. 11), which is consistent with a location of an aph VIII expression cassette added in the artificially synthesized chloroplast genome during design. In RFLP-Southern Blot results of all transformants, only one band appears, indicating that transformed strains have been homogenized, and the artificially totally synthesized chloroplast genome has completely replaced the wild-type chloroplast genome.

### 4. Homogenization of Transformants

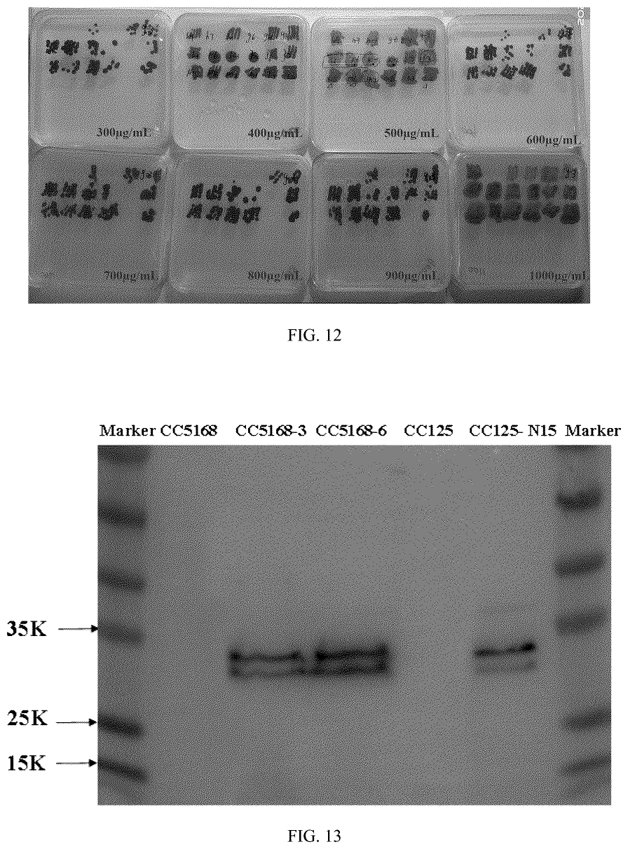

Transformants identified by PCR were transferred onto resistant plates with streptomycin concentrations of 150 μg/mL, 300 μg/mL, 400 μg/mL, 500 μg/mL, 600 μg/mL, 700 μg/mL, 800 μg/mL, 900 μg/mL and 1000 μg/mL, and continued to be cultured in an incubator with a photoperiodic ratio of 16:8 (as shown in FIG. 12). Results show that when the streptomycin concentration is 300-900 μg/mL, the algal cells can grow normally, while the morphology of the algal cells with 1000 μg/mL changes significantly. 300, 600, and 900 μg/mL algal cell samples were taken and sent to a commercial company for sequencing.

### 5. Western Blot Verification

A photo-deficient algal strain CC5168 and transformants CC5168-3 and CC5168-6 were cultured to a logarithmic phase, and a total protein of algal cells was extracted. Finally, protein samples were suspended in a protein sample buffer solution (60 mM Tris pH 6.8, 2% (w/v) lauryl sodium sulfate, 10% (v/v) glycerol, 0.01% (w/v) bromophenol blue). The protein was separated with triglycine SDS-PAGE. Gel before immunoassay was subjected to Western Blot on a nitrocellulose membrane, by using HRP linked mouse antistreptococcic monoclonal antibody (1:5000, including 5% (w/v) BSA and milk powder (blocking agent) in TBS) or rabbit anti-glucose. Subsequently, the buffer solution was blocked by the antibody (1:5000 in a closed buffer solution) through HRP linked goat anti-Rabbit IgG (1:10000). A Pierce ECL Western Blot substrate and a Fusion Fx7 CCD camera were used for observation.

In order to study the protein expression in the artificial chloroplast genome of Chlamydomonas reinhardtii, HA tags were added to rps4 (33.3 Kd) and atpI (29.4 Kd) proteins. A total protein of Chlamydomonas reinhardtii CC5168 and transformants CC5168-3 and CC5168-6 thereof was extracted, and subjected to protein hybridization blot analysis with an HA monoclonal antibody. Results are shown in FIG. 13, where photo-deficient CC5168 and wild-type CC125 serve as a control group, CC5168-3 and CC5168-6 are positive transformed algal strains of CC5168, and CC125-N15 is a positive transformed algal strain of a CC125 synthetic genome. Experimental results show that the Chlamydomonas reinhardtii CC5168 has no hybridization blot, and the transformants CC5168-3 and CC5168-6 can be hybridized to an rps4 protein with a size of 33.3 Kd and an atpI protein with a size of 29.4 Kd (FIG. 13), indicating that marker proteins on the totally chemically synthesized artificial chloroplast genome of Chlamydomonas reinhardtii can be expressed normally, and the totally chemically synthesized genome has transcriptional and translational function activity.

### 6. Analysis of Photosynthetic Ability of Repair Photo Mutant Strain

### 1) Growth Curve

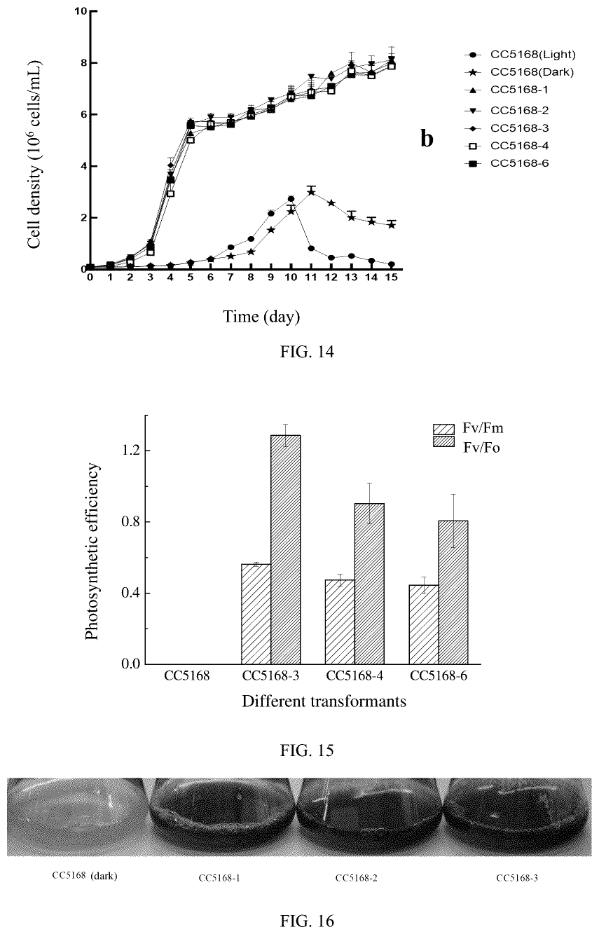

CC5168 and positive transformed algal strains CC5168-1, CC5168-2, CC5168-3, CC5168-4 and CC5168-6 were inoculated to a TAP liquid medium, under conditions of 25° C., 30 μE/m²/s, light for 16 h, and dark for 8 h. In addition, CC5168 was inoculated to total dark as a control and cultured for 0 d, 1 d, 2 d, 3 d, 4 d, 5 d, 6 d, 7 d, 8 d, 9 d, 10 d, 11 d, 12 d, 13 d, 14 d and 15 d, respectively. OD750 values at different culture time were measured, and growth curves were plotted, as shown in FIG. 14. The growth status of the transformants was consistent with that of the receptor strains, indicating that the artificial chloroplast genome of Chlamydomonas reinhardtii could maintain the normal growth of algae cells. Chlamydomonas reinhardtii CC5168 was a psbH gene-deleted mutant strain that could not undergo photosynthesis and could only grow slowly under a condition of total dark, and had the number of cells reached 3×10⁴ cells/mL after 11 days of cultivation, however, its transformant could grow under continuous light conditions of 22°° C. and 30 μmol m⁻² s⁻¹, and restored the growth status of the wild type, indicating that after the synthesized artificial chloroplast genome of Chlamydomonas reinhardtii entered Chlamydomonas reinhardtii CC5168 cells, the deletion mutation of psbH was repaired and the photoautotrophic function of the receptor strains was restored (FIG. 14).

### 2) Photosynthetic Efficiency

Algae cells cultured to a logarithmic phase were inoculated into a fresh TAP medium with an initial cell concentration of 4×10⁴ cells/mL, and sampled daily after inoculation, and concentrations of cultured algae were calculated by using a cell counter until it grew to a plateau. Growth curves of different algae strains were plotted by using cell concentration obtained by counting as ordinate, and using growth time as abscissa. A Chlorophyll fluorometer PhytoPAM was used to measure Fv/Fm, Fv/Fo, and ΔF/Fm′ of algae strains, refer to the PhytoPAM instructions for use for specific measurement methods. Results are shown in FIG. 15.

Research results show that under the continuous light conditions of 22° C. and 30 μmol m⁻² s⁻¹, the Chlamydomonas reinhardtii CC5168 cannot undergo normal photosynthesis, and its transformants CC5168-2, 5168-3, 5168-4 and 5168-6 can undergo normal photosynthesis, indicating that the photosynthetic ability of Chlamydomonas reinhardtii CC5168 is restored (FIGS. 15 and 16).

In summary, the present invention provides a method for synthesis, assembly and function test of an artificial chloroplast genome of Chlamydomonas reinhardtii. According to the present invention, rational design has been carried out on the Chlamydomonas reinhardtii chloroplast genome for the first time, and total artificial synthesis of the Chlamydomonas reinhardtii chloroplast genome is proposed. By using the method of synthetic biology, all nucleic acid segments are constructed from chemically synthesized nucleic acid sequences. By using totally chemically synthesized chloroplast genome segments, total chemical de novo synthesis and assembly of a chloroplast genome are achieved in a yeast-bacterium system. Then, a totally chemically synthesized chloroplast genome is transformed into Chlamydomonas cells to replace the original chloroplast genome through various technical means, which works normally, and has been verified, fulfilling biological functions of the totally chemically synthesized chloroplast genome. The method can be widely applied in gene editing of the Chlamydomonas reinhardtii chloroplast genome and production of antibody drugs and the like, and has huge commercial advantages and broad market prospects. Meanwhile, according to the present invention, the Chlamydomonas reinhardtii chloroplast genome is an efficient platform for carrying out synthetic biology operation, the de novo design, total chemical synthesis, in vitro assembly and identification of the genome thereof provide a new solution for the rational design, modification and reconstruction of a photosynthetic system of photosynthetic organisms, improvement of photosynthetic efficiency of crops, and solving of agricultural crisis such as food security.

It should be understood that the application of the present invention is not limited to the above examples. For those of ordinary skill in the art, improvements or transformations can be made according to the above description, and all these improvements and transformations should fall within the protection scope of the claims attached to the present invention.

1-10. (canceled)

11. A method for synthesis, assembly, and function test of an artificial chloroplast genome of Chlamydomonas reinhardtii, the method comprising de novo design, total chemical synthesis, assembly and function verification of an artificial chloroplast genome of Chlamydomonas reinhardtii, wherein a nucleotide sequence of the artificial chloroplast genome of Chlamydomonas reinhardtii is obtained by designing and modifying a nucleotide sequence of a wild-type Chlamydomonas reinhardtii chloroplast genome and adding a BAC vector backbone, a streptomycin resistance gene aadA, a paromomycin resistance gene aph VIII and an HA tag.

12. The method of claim 11, wherein the nucleotide sequence of the artificial chloroplast genome of Chlamydomonas reinhardtii has a full length of 221,372 bp.

13. The method of claim 11, wherein the artificial chloroplast genome of Chlamydomonas reinhardtii is divided into 44 primary segments when being de novo designed, each of the primary segments having 120 bp homologous recombination sequences at two ends.

14. The method of claim 13, wherein the primary segments are all synthesized through a chemical method.

15. The method of claim 11, wherein the BAC vector backbone has a length of 11,060 bp, and an insertion site is located at 205,535 bp of the wild-type Chlamydomonas reinhardtii chloroplast genome; the streptomycin resistance gene aadA has a length of 1,630 bp, and an insertion site is located between 173,174 bp and 173,175 bp of the wild-type Chlamydomonas reinhardtii chloroplast genome; the streptomycin resistance gene aadA has a length of 2,287 bp, and an insertion site is located between 71,064 bp and 71,065 bp of the wild-type Chlamydomonas reinhardtii chloroplast genome; a 3×HA-1 tag is located behind atpI, and an insertion site is located between 170,786 bp and 170,787 bp of the wild-type Chlamydomonas reinhardtii chloroplast genome; a 3×HA-2 tag is located behind rps4, and an insertion site is located between 33,292 bp and 33293 bp of the wild-type Chlamydomonas reinhardtii chloroplast genome.

16. The method of claim 11, wherein the synthesis and assembly of the artificial chloroplast genome of Chlamydomonas reinhardtii comprises the following steps:
connecting the 44 primary segments of the de novo designed artificial chloroplast genome of Chlamydomonas reinhardtii to a pUC18 vector, respectively;
co-transforming a BAC vector segment, a screening marker segment, a 7-35 bridging segment, a seg44 segment, segments seg35-seg43, a seg1 segment, and segments seg3-seg7 into a yeast BY4741, and screening by using a screening medium SC-URA to obtain a yeast strain-1 containing an intermediate plasmid 1;
co-transforming a pRS415 vector segment and segments seg7-seg21 into a yeast BY4742, screening by using a screening medium SC-LEU, using a kanMX segment to replace a Met gene on a genome with a yeast strain obtained by screening for Met knockout, and using an SC-LEU+G418 medium for screening to obtain a yeast strain-2 containing an intermediate plasmid 2;
co-transforming a pRS411 vector segment and segments seg22-seg35 into a yeast BY4741, and using a screening medium SC-MET for screening to obtain a yeast strain-3 containing an intermediate plasmid 3;
hybridizing the yeast strain-2 and the yeast strain-3, and screening through an SC-LEU-MET plate; then carrying out spore production and spore division, and screening through an SC-LEU plate and an SC-MET plate; then hybridizing with a yeast SZU-JDY19 and a yeast SZU-JDY20; then hybridizing with the yeast strain-1 containing the intermediate plasmid 1, and screening through an SC-LEU-MET-URA plate; transforming an SZU-ZLP012 plasmid of a strain obtained by screening, and screening through an SC-URA-LEU-MET-HIS plate; and
inducing I-SceI gene expression in the SZU-ZLP012 plasmid by using a galactose, cleaving an I-SceI site to linearize 3 intermediate plasmids, and then obtaining a yeast strain containing a complete artificial chloroplast genome of Chlamydomonas reinhardtii through homologous recombination in a yeast cell.

17. The method of claim 16, wherein a nucleotide sequence of the intermediate plasmid 1 has a full length of 92,477 bp and contains nucleotide sequences of 1-33,292 bp and 159,554 bp-205,535 bp of the wild-type Chlamydomonas reinhardtii chloroplast genome, and a BAC backbone sequence is added at 205,535 bp of the wild-type Chlamydomonas reinhardtii chloroplast genome; a nucleotide sequence of the intermediate plasmid 2 has a full length of 81,426 bp and contains nucleotide sequences of 28,513 bp-102,566 bp of the wild-type Chlamydomonas reinhardtii chloroplast genome, and pRS406 vector sequences are connected at head and tail interfaces of genome nucleotide sequences; a nucleotide sequence of the intermediate plasmid 3 has a full length of 72,377 bp and contains nucleotide sequences of 97,668 bp-164,450 bp of the wild-type Chlamydomonas reinhardtii chloroplast genome, and pRS411 vector sequences are connected at head and tail interfaces of genome nucleotide sequences.

18. The method of claim 11, wherein the function test of the artificial chloroplast genome of Chlamydomonas reinhardtii comprises the following steps:
transferring the artificial chloroplast genome of Chlamydomonas reinhardtii into a chloroplast of Chlamydomonas reinhardtii CC5168 by particle bombardment, and obtaining positive transformants by streptomycin screening, PCR and Southern Blot screening;
carrying out homogenization screening on the positive transformants by using gradient streptomycin resistance concentration; and
carrying out Western Blot experiment on the positive transformants for verification of protein expression; and detecting a growth curve of the positive transformants and photosynthesis of a repair mutant strain.

19. The method of claim 18, wherein detection segments for identification and positive screening of the positive transformants are aphVIII, BAC-seg44, and BAC-seg1, respectively.

20. The method of claim 18, wherein a probe used for Southern Blot verification of the positive transformants is obtained by amplification of aph VIII-F1/R1 and psaA-F/R primers, nucleotide sequences of the aph VIII-F1/R1 primer being shown in SEQ ID NO.1 and SEQ ID NO.2, and nucleotide sequences of the psaA-F/R primer being shown in SEQ ID NO.3 and SEQ ID NO.4.
